(19) United States
(12) Patent Application Publication
(10) Pub. No.: US 2016/0022324 A1
(43) Pub. Date: Jan. 28, 2016

(54) INTERSPINOUS FUSION IMPLANT

(71) Applicant: ANT TECHNOLOGY, Yongin-si (KR)

(72) Inventors: Hong Won YOON, Yongin-si (KR); Eun Mi JUNG, Seoul (KR); Kwang Hoon LEE, Pyeongtaek-si (KR)

(21) Appl. No.: 14/876,127

(22) Filed: Oct. 6, 2015

Related U.S. Application Data
(63) Continuation-in-part of application No. PCT/KR2014/008003, filed on Aug. 28, 2014.

(30) Foreign Application Priority Data
Nov. 13, 2013 (KR) 10-2013-0137594
Jun. 5, 2015 (KR) 10-2015-0079699

(51) Int. Cl. A61B 17/70; A61F 2/44

(52) U.S. Cl. CPC A61B 17/7056; A61F 2/4455

(57) ABSTRACT

Provided is an interspinous fusion implant that is capable of fixing a vertebral body having a lesion by using hooking between spinous processes through a minimal incision without using a pedicle screw. The interspinous fusion implant includes an upper hooking member installed to be hooked with one spinous process and a lower hooking member installed to be engaged with another adjacent spinous process. The upper hooking member and the lower hooking member are coupled to each other and then fixed to each other through a fixing bolt after a distance between vertebral bodies having a lesion is reduced by using a separate mechanism.

### CROSS-REFERENCE TO RELATED APPLICATIONS

The instant application is a continuation-in-part application of international application PCT/KR2014/008003 filed on Aug. 28, 2014, which claims the benefit of priority from Korean Patent Application No. 10-2013-0137594 filed on Nov. 13, 2013, and the instant application further claims priority to Korean Patent Application No. 10-2015-0079699 filed on Jun. 5, 2015, the entire contents of which are incorporated herein by reference.

### BACKGROUND

The present disclosure relates to an interspinous fusion implant, and more particularly, to an interspinous fusion implant that is capable of fixing a vertebral body having a lesion by pulling two adjacent spinous processes by employing a hooking technique and making a minimum incision without using a pedicle screw.

In general, the spine is made up of 33 bones to support the human body and protect spinal nerves. The spine is made of 7 cervical vertebrae in the neck, 12 thoracic vertebrae in the thorax, 5 lumbar vertebrae in the lower back, 5 sacral vertebrae in the sacrum, and 4 coccygeal vertebrae in the coccyx.

The spine is made of a plurality of independent vertebral bodies that are connected to each other by joints. Here, a soft disc (or cartilage) is filled between the joints to connect the joints to each other. The spine having the above-described configuration absorbs shock, supports various postures, and protects organs within the human body.

The spine has a complex structure composed of various anatomical elements and also has a structure that provides flexibility and stability to the human body.

The spine is constituted of the vertebrae, and each of the vertebral bodies has a cylindrical shape.

The vertebral bodies which are adjacent to each other have opposite sides connected to each other, and simultaneously, are separated by an intervertebral disc (or disc) made of a fibrocartilaginous material.

Also, the vertebral bodies are connected to each other by complex ligament tissues that interact with each other to restrict excessive movement and provide stability.

The vertebra has a thick lateral portion that is called a lateral mass. Each of the lateral masses includes facets on upper and inner portions thereof.

The facet of one vertebra is coupled to the inner facet of the next adjacent vertebra. The coupling of the vertebrae is a so-called “facet joint”.

A stable spine is important to prevent incapacitating pain, progressive deformity, and/or neurological compromise, which emasculate the functions of the spine.

The present surgical treatment method for a ligament injury of the spine includes the removal of facet joint capsules and arthrodesis of the joints.

In this case, in the treatment for instability of the lower cervical vertebra, a screw that extends through the lateral mass of the adjacent vertebra is generally used.

A limitation that arises with this technique is that the spinal nerves may be damaged when the screw is inserted into the lateral mass.

### SUMMARY

Embodiments provide an interspinous fusion implant that is capable of fixing a vertebral body having a lesion by using hooking between spinous processes through a minimum incision without using a pedicle screw.

Embodiments also provide an interspinous fusion implant in which a lower hooking member is coupled to an upper hooking member through liner movement thereof without an unnecessary operation such as the rotation of the lower hooking member when a vertebral body having a lesion is fixed by using hooking between the upper and lower spinous processes through a minimum incision without using a pedicle screw.

In one embodiment, an interspinous fusion implant includes: an upper hooking member installed to be hooked with one spinous process; and a lower hooking member installed to be hooked with another adjacent spinous process, wherein the upper hooking member and the lower hooking member are coupled to each other and then fixed to each other through a fixing bolt after a distance between vertebral bodies having a lesion is reduced by using a separate mechanism.

In another embodiment, an interspinous fusion implant includes: an upper hooking member hooked with an upper spinous process; and a lower hooking member hooked with an adjacent lower spinous process, wherein gapping of the upper and lower spinous processes in opposite directions is prevented, the upper hooking member includes an upper hook for pulling the upper spinous process downward and an insertion part disposed with a predetermined length on a lower portion of the upper hook, and the lower hooking member includes a lower hook for pulling the lower spinous process upward, a guide frame disposed on an upper portion of the lower hook and in which the insertion part is slidably coupled to an inner accommodation part that is longitudinally defined therein, and a fastening member passing from one side surface of the guide frame and coupled to the guide frame, the fastening member being closely attached to the insertion part coupled to the inner accommodation part to firmly fasten the insertion part.

In further embodiment, an interspinous fusion implant includes: an upper hooking member hooked with an upper spinous process; and a lower hooking member hooked with an adjacent lower spinous process, wherein gapping of the upper and lower spinous processes in opposite directions is prevented, the upper hooking member includes an upper hook for pulling the upper spinous process downward, a first coupling part that is recessed to be stepped downward by a predetermined length at one side of the upper hook, a plurality of fastening members inserted in a line along a longitudinal direction of the first coupling part, and the lower hooking member includes a lower hook for pulling the lower spinous process upward and a second coupling part that is recessed to be stepped upward by a predetermined length at one side of the lower hook, wherein a slot-shaped accommodation groove is defined in a center of the second coupling part along a longitudinal direction to divide the second coupling part into a left and a right part, and the second coupling part is guide by the plurality of fastening members coupled in sequence to the slot-shaped accommodation groove, and is engaged with the first coupling part.

In further embodiment, an interspinous fusion implant includes: an upper hooking member hooked with an upper spinous process; and a lower hooking member hooked with an adjacent lower spinous process, wherein gapping of the upper and lower spinous processes in opposite directions is prevented, the upper hooking member includes an upper hook for pulling the upper spinous process downward and an upper body extending by a predetermined length from a lower portion of the upper hook and having a slot into which the lower hooking member is coupled, the lower hooking member includes a lower hook for pulling the lower spinous process upward and a lower body extending by a predetermined length from an upper portion of the lower hook, having a long hole in a longitudinal direction of the body, and coupled to the slot of the upper body, wherein the interspinous fusion implant further includes: a fixing bolt passing through the upper body and the long hole of the lower body coupled to the slot of the upper body and coupled and fastened to fix the upper and lower hooking members; and a rotation preventing member disposed on the same line as the fixing bolt along a longitudinal direction of the upper body to pass through the long hole of the lower body together with the fixing bolt and to be coupled to prevent the coupled lower body from rotating with respect to the fixing bolt as an axis and allow the lower body to linearly move when the coupled lower body moves within the slot of the upper body.

The details of one or more embodiments are set forth in the accompanying drawings and the description below. Other features will be apparent from the description and drawings, and from the claims.

### BRIEF DESCRIPTION OF THE DRAWINGS

FIG. 1 is an exploded perspective view of an interspinous fusion implant according to a first embodiment.

FIG. 2 is a perspective view illustrating an assembled state of the interspinous fusion implant of FIG. 1.

FIG. 3A is a view illustrating a state in which an upper hooking member of the interspinous fusion implant is hooked with a spinous process according to the first embodiment.

FIG. 3B is a view illustrating a state in which a lower hooking member of the interspinous fusion implant is hooked with a spinous process according to the first embodiment.

FIG. 3C is a view illustrating a state in which the interspinous fusion implant reduces the distance between one spinous process having a lesion and another spinous process according to the first embodiment.

FIG. 3D is a view illustrating a state in which the interspinous fusion implant is fixed to the one spinous process having the lesion and the other spinous process according to the first embodiment.

FIG. 4 is a perspective view of an interspinous fusion implant according to a second embodiment.

FIG. 5 is a horizontal cross-sectional view of FIG. 4.

FIG. 6 is a perspective view and horizontal cross-sectional view of an interspinous fusion implant having a different shape according to the second embodiment.

FIG. 7A is a view illustrating an example of a state in which the interspinous fusion implant is hooked between adjacent spinous processes according to the second embodiment.

FIG. 7B is a view illustrating an example of a state in which the interspinous fusion implant is hooked between the adjacent spinous processes and then coupled to a plate according to the second embodiment.

FIG. 8 is an exploded perspective view of the interspinous fusion implant according to the second embodiment.

FIG. 9A is a view illustrating an example of a process in which an interspinous fusion implant is assembled according to a third embodiment.

FIG. 9B is a side view illustrating a process in which the interspinous fusion implant is assembled according to the third embodiment.

FIG. 9C is a view illustrating an example of a state in which the interspinous fusion implant is hooked with adjacent spinous processes according to the third embodiment.

FIG. 10A is a perspective view illustrating a state in which a spacer is attached to the interspinous fusion implant according to the third embodiment.

FIG. 10B is a view illustrating an example of a state in which the interspinous fusion implant of FIG. 10A is hooked between adjacent spinous processes.

FIGS. 11A and 11B are exploded perspective views of an interspinous fusion implant according to a fourth embodiment.

FIG. 11C is a front view of the interspinous fusion implant according to the fourth embodiment.

FIG. 11D is a perspective view of the interspinous fusion implant according to the fourth embodiment.

FIG. 11E is a view illustrating an example of a state in which the interspinous fusion implant is hooked with adjacent spinous processes according to the fourth embodiment.

### DETAILED DESCRIPTION OF THE EMBODIMENTS

Since the present disclosure may implement diverse modifications and have many embodiments, specific embodiments are illustrated in the drawings and are described in detail in the detailed description.

However, it is not the intention to limit the present disclosure to specific embodiments and it should be understood that the present disclosure covers all modifications, equivalents, and replacements within the idea and technical scope of the present disclosure.

In the description of the drawings, like reference numerals refer to like elements throughout. It will be understood that although the terms “first” and “second” are used herein to describe various elements, these elements should not be limited by these terms. The terms are only used to distinguish one component from other components.

For example, the word “and/or” means that one or more or a combination of relevant constituent elements is possible.

Unless terms used in the present disclosure are defined differently, the terms may be construed to have a meaning known to those skilled in the art.

Terms such as those that are generally used and are defined in dictionaries should be construed as having meanings matching contextual meanings in the art. In this description, unless defined clearly, terms are not to be interpreted as having ideally or excessively formal meanings.

Hereinafter, an interspinous fusion implant according to embodiments will be described in detail with reference to the accompanying drawings.

### First Embodiment

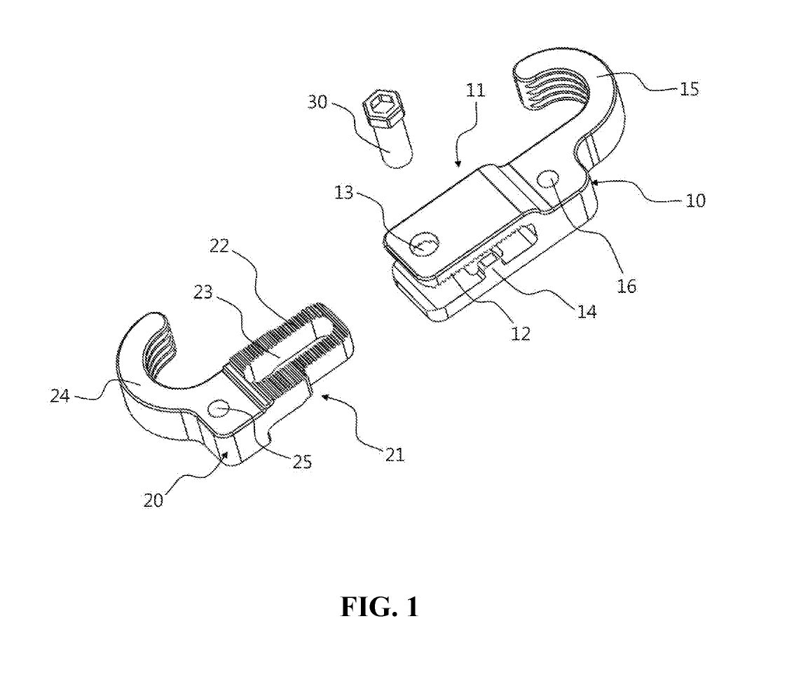

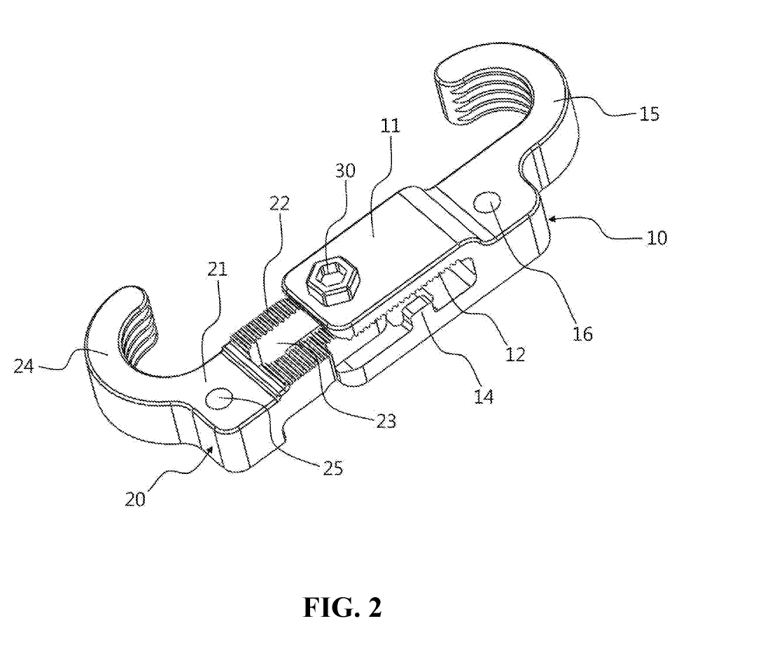

FIG. 1 is an exploded perspective view of an interspinous fusion implant according to a first embodiment, and FIG. 2 is a perspective view illustrating an assembled state of the interspinous fusion implant of FIG. 1.

As illustrated in FIGS. 1 and 2, an interspinous fusion implant according to a first embodiment includes an upper hooking member 10 configured to be hooked with a spinous process of a spine having a lesion and a lower hooking member 20 that is coupled to the upper hooking member 10 to easily rotate and move inside the upper hooking member 10. The upper hooking member 10 and the lower hooking member 20 are fixed to each other by using a fixing bolt 30 so that the gap between the one spinous process of the spine having the lesion and the other adjacent spinous process is maintained in a narrowed state.

The upper hooking member 10 includes an upper body 11 having a “ [symbol] ” shape, and an upper latch 12 is disposed on one surface of the inside of the upper body 11 to fix the position of the lower hooking member 20. A fixing hole 13, into which the fixing bolt 30 is inserted to fix the positions of the upper and lower hooking members 10 and 20, is defined in the upper hooking member 10. A plurality of rotation preventing protrusion 14, for preventing the lower hooking member 20 coupled to the inside of the upper hooking member 10 from rotating, are disposed inside the upper body 11, and an upper hook 15 is integrated with one side of the upper body 10 so that the upper hooking member 10 is hooked with the spinous process of the spine in which the lesion is present.

The lower hooking member 20 includes a lower body 21 coupled to the inside of the upper body 10 having the “ [symbol] ” shape and a lower latch 22 disposed on one surface of the inside of the lower body 21 so that the lower latch 22 is engaged with the upper latch 12 and fixed in position. A long hole 23 is defined in the lower body 21 and coupled with the fixing bolt 30 to allow the lower body 21 to move and rotate inside the upper body 10. A lower hook 24 is integrated with the lower body 21 so that the lower hooking member 20 is hooked with the other spinous process that is adjacent to the spinous process with which the upper hooking member 10 is hooked.

The lower hooking member 20 as configured above is inserted into the upper hooking member 10 and has the fixing bolt 30 inserted and coupled to the fixing hole 13 of the upper hooking member 10 and the long hole 23 of the lower hooking member 20. The fixing bolt 30 is supported on one side of the long hole 23 of the lower hooking member 20 that is inserted into and coupled to the inside of the upper hooking member 10 to rotate or move in a longitudinal direction of the upper hooking member 10.

Also, non-explained reference numerals 16 and 25 in the drawings represent an upper hooking hole 16 and a lower hooking hole 25, which are defined in the upper body 11 and the lower body 21, respectively.

The upper hooking hole 16 and the lower hooking hole 25 are coupled to separate mechanisms to hook the upper hooking member 10 with the one spinous process and the lower hooking member 20 with the other adjacent spinous process, respectively. Also, the upper hooking hole 16 and the lower hooking hole 25 are used to reduce the distance between the hooked upper and lower hooking members 10 and 20.

The upper hooking hole 16 and the lower hooking hole 25 do not have to be defined in the upper hooking member 10 and the lower hooking member 20 and may be selectively defined by a manufacturer for convenience in surgical operation.

A process in which the interspinous fusion implant according to the first embodiment is installed on the spinous process having the lesion will be described with reference to the accompanying drawings.

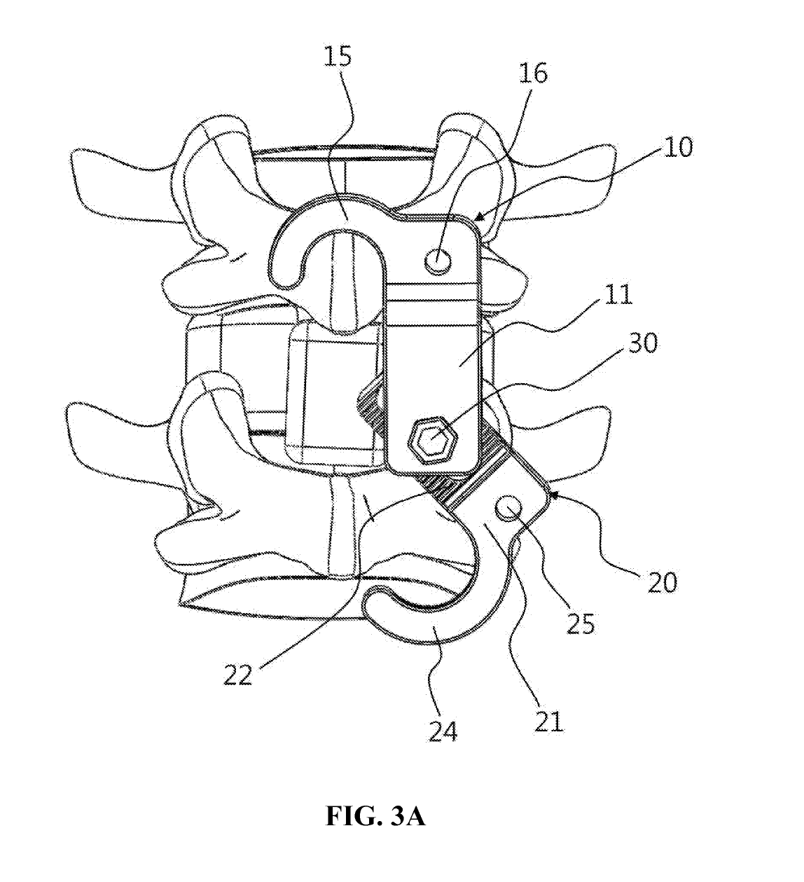

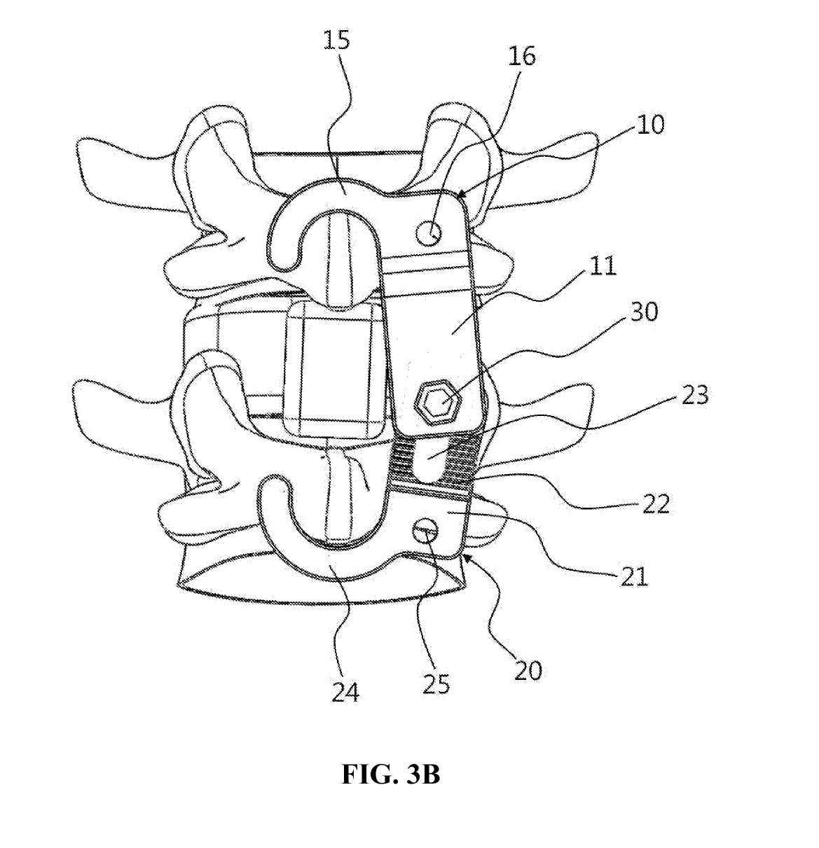

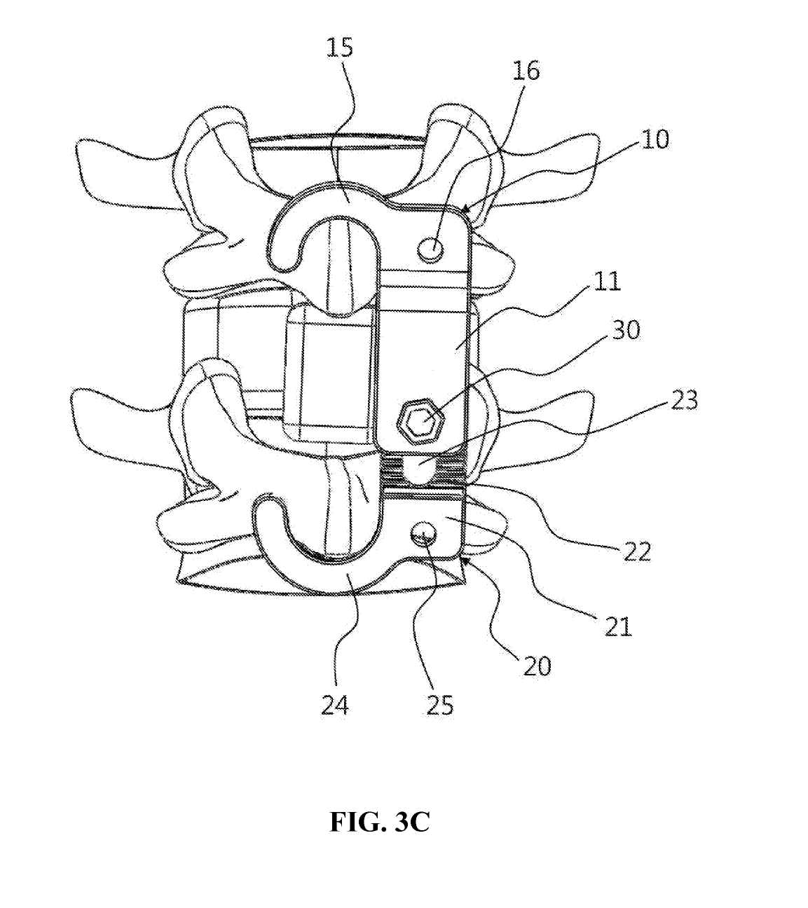

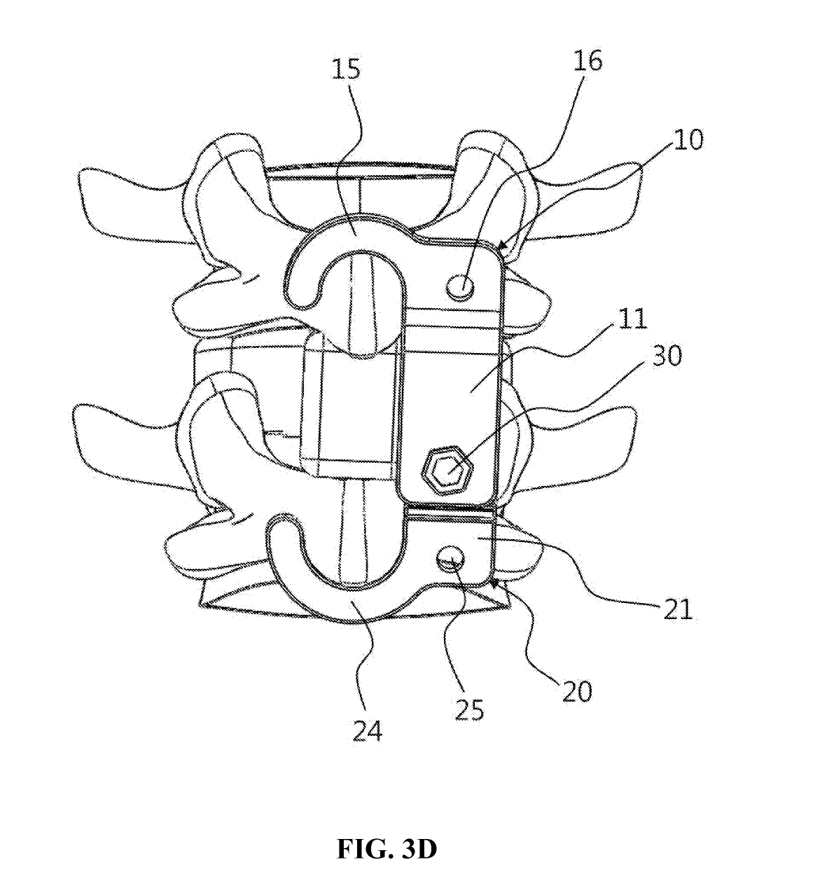

FIG. 3A is a view illustrating a state in which the upper hooking member of the interspinous fusion implant is hooked with the spinous process according to the first embodiment, FIG. 3B is a view illustrating a state in which the lower hooking member of the interspinous fusion implant is hooked with the spinous process according to the first embodiment, FIG. 3C is a view illustrating a state in which the interspinous fusion implant reduces the distance between one spinous process having the lesion and another spinous process according to the first embodiment, and FIG. 3D is a view illustrating a state in which the interspinous fusion implant is fixed to the one spinous process having the lesion and the other spinous process according to the first embodiment.

First, the skin on a portion at which a spine having a lesion is disposed is incised. Here, the skin is incised by a length sufficient to insert the interspinous fusion implant into the human body.

A bone block (not shown) is installed between one spinous process of a vertebral body having a lesion and an adjacent spinous process, in the state in which the skin is incised.

The bone block is formed of a material such as an autogenous bone, an allogenous bone, or an artificial bone. Only a safe and sterilized product certified by the Korean Food and Drug Administration may be used as the bone block to prevent the occurrence of infection during surgery.

The separate mechanisms are fixed to the upper hooking hole 16 and the lower hooking hole 25 which are defined in the upper hooking member 10 and the lower hooking member 20 on the bone block in a state in which the bone block is disposed between the spinous processes. The upper hook 15 of the upper hooking member 10 is disposed on the upper spinous process, and the lower hook 24 of the lower hooking member 20 is disposed on the other adjacent spinous process by using the fixed mechanisms.

As illustrated in FIG. 3B, when the lower hook 24 is disposed, the lower hooking member 20 is withdrawn and rotated to one side of the upper hooking member 10 to adjust the length and position thereof and is then hooked with the spinous process.

The distance between the upper hooking member 10 and the lower hooking member 20 is reduced by using the mechanisms fixed to the upper hooking hole 16 and the lower hooking hole 25 so that the upper hooking member 10 hooked with the spinous process and the lower hooking member 20 hooked with the adjacent spinous process are closely attached to each other.

When the distance between the upper hooking member 10 and the lower hooking member 20 is reduced, the lower body 20 is lifted slightly so that the lower latch 22 disposed on the lower body 21 of the lower hooking member 20 is engaged with the upper latch 12 disposed on the upper body 10.

The lower body 21 is inserted into the upper body 11 to prevent the lower body 21 from rotating in the upper body 11 by the rotation preventing protrusion 14 disposed on the upper body 11.

Here, the lower latch 22 is engaged with the upper latch 12 to fix the position of the lower hooking member 20. Also, the fixing bolt 30 installed in the upper hooking member 10 and the lower hooking member 20 is rotated to fix the positions of the upper hooking member 10 and the lower hooking member 20.

As illustrated in FIG. 3D, the bone block is very closely attached between the spinous processes through the above-described fixation and is thus firmly fixed.

Also, since the fixing bolt 30 is fixed, the upper hooking member 10 and the lower hooking member 20 may be completely fixed without movement to stably fix the corresponding portion of the spine.

### Second Embodiment

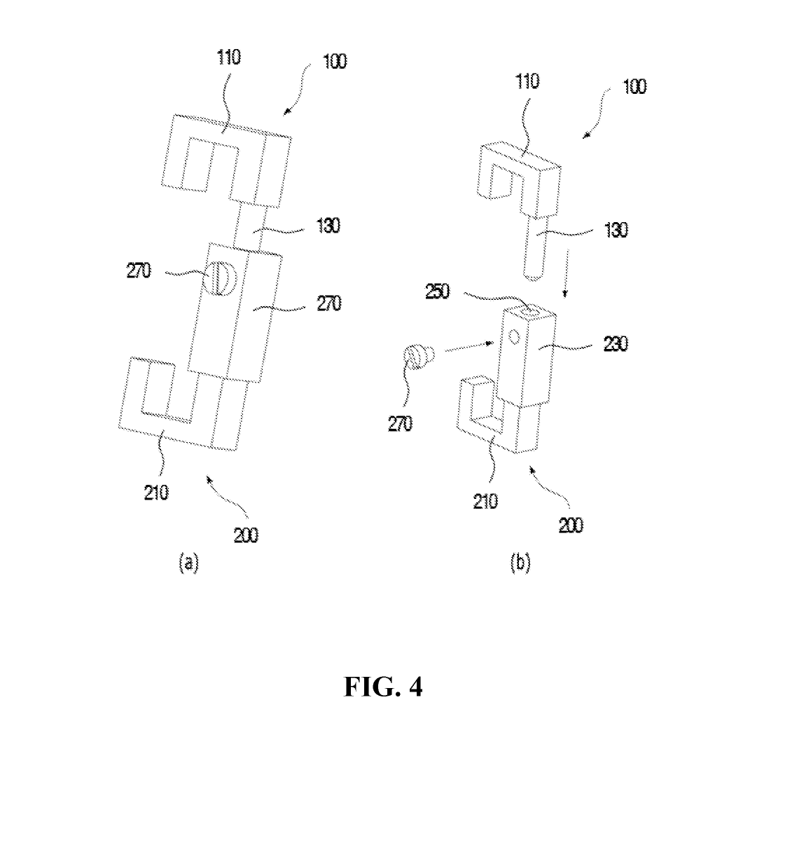

Referring to FIGS. 4 to 8, an interspinous fusion implant according to a second embodiment includes an upper hooking member 100 hooked with an upper spinous process sp1 of a spine having a lesion and a lower hooking member 200 hooked with an adjacent lower spinous process sp2, wherein gapping of the upper and lower spinous processes sp1 and sp2 in opposite directions is prevented. The upper hooking member 100 includes an upper hook 110 for pulling the upper spinous process sp1 downward and an insertion part 130 disposed on one side of a lower portion the upper hook 110 and having a predetermined length, and the lower hooking member 200 includes a lower hook 210 for pulling the lower spinous process sp2 upward, a guide frame 230 disposed on one side of an upper portion of the lower hook 210 and in which the insertion part 130 is slidably coupled to an inner accommodation part 250 that is longitudinally defined therein, and a fastening member 270 passing from one side surface of the guide frame 230 and coupled to the guide frame 230 and closely attached to the insertion part 130 coupled to the inner accommodation part 250 to firmly fasten the insertion part 130.

Since the insertion part 130 is slidably coupled to the inner accommodation part 250 of the guide frame 230, rotation of the lower or upper hooking member may be prevented, unlike the first embodiment. Thus, operation time may be reduced, and concern about neural damage around an operation site may be alleviated.

Each of the upper hook 110 and the lower hook 210 may have various shapes such as a “ [symbol] ” shape or an arc shape if each of the upper and lower hooks 110 and 210 has a structure that is capable of being hooked with each of the spinous processes sp1 and sp2.

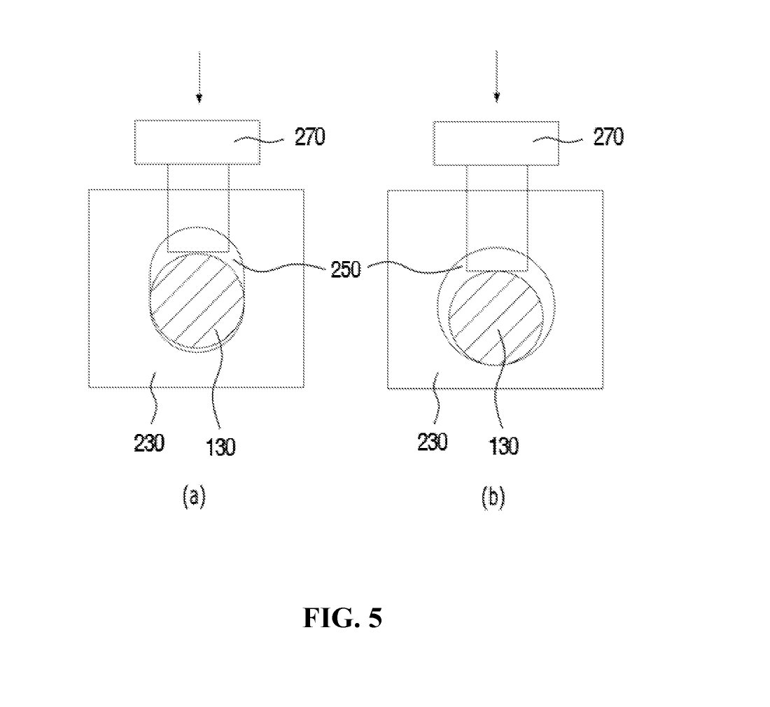

Referring to FIG. 5, an inner circumferential surface of the guide frame 230 has a circular or oval shape, and an outer circumferential surface of the insertion part 130 has a circular shape. FIG. 5A illustrates the guide frame 230 with the inner circumferential surface having an oval shape, and FIG. 5B illustrates the guide frame 230 with the inner circumferential surface having a circular shape. Since the fastening member 270 passes through a hole h to firmly fasten a side surface of the insertion part 130, the upper and lower hooking members 100 and 200 are fixed.

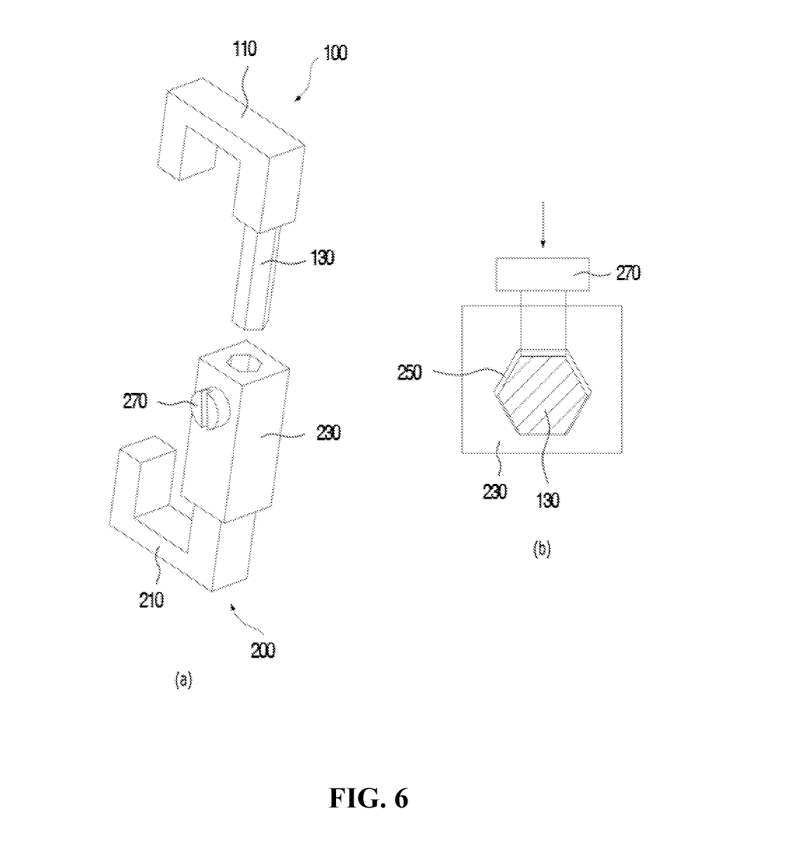

Referring to FIG. 6, the inner circumferential surface of the guide frame 230 and the outer circumferential surface of the insertion part 130 may have polygonal shapes corresponding to each other. In the current embodiment, a hexagonal shape is provided as an example.

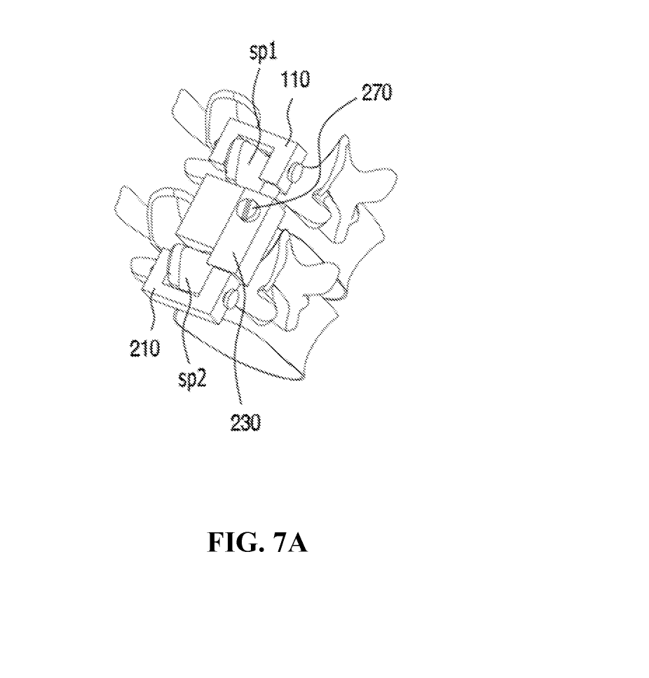

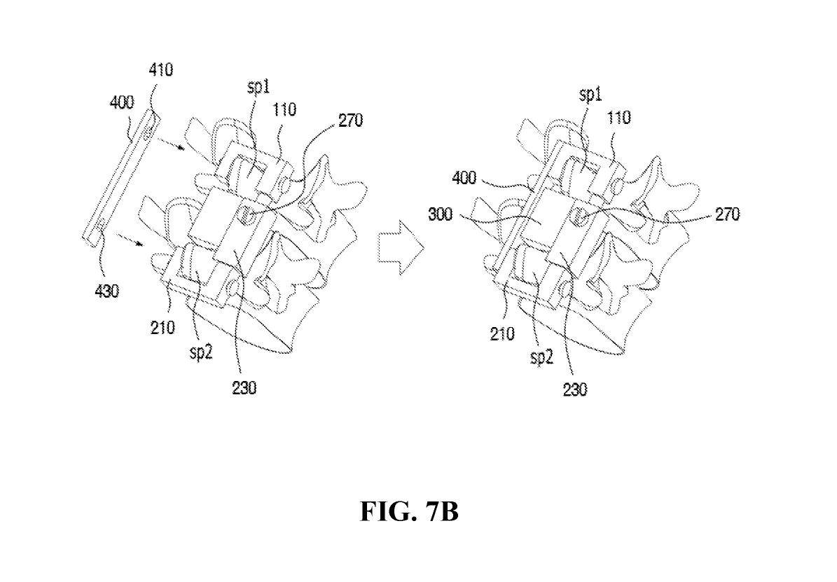

Referring to FIGS. 7A and 7B, the interspinous fusion implant further includes a spacer 300 parallely attached to a side surface of the guide frame 230 and disposed between the upper and lower spinous processes sp1 and sp2 to maintain a minimum distance between the spinous processes sp1 and sp2. The spacer 300 may be usefully used when it is necessary to maintain a distance between the upper and lower spinous processes sp1 and sp2.

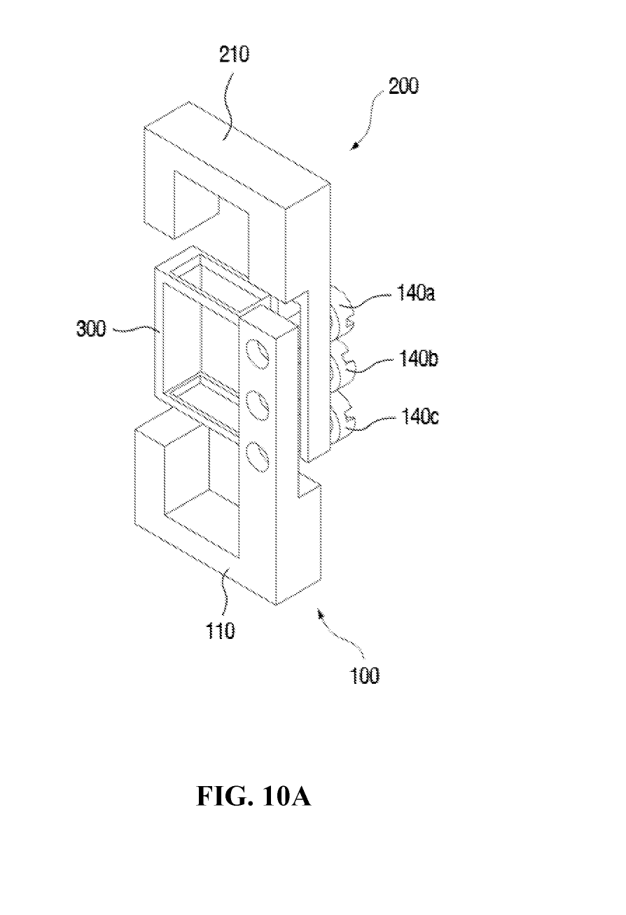

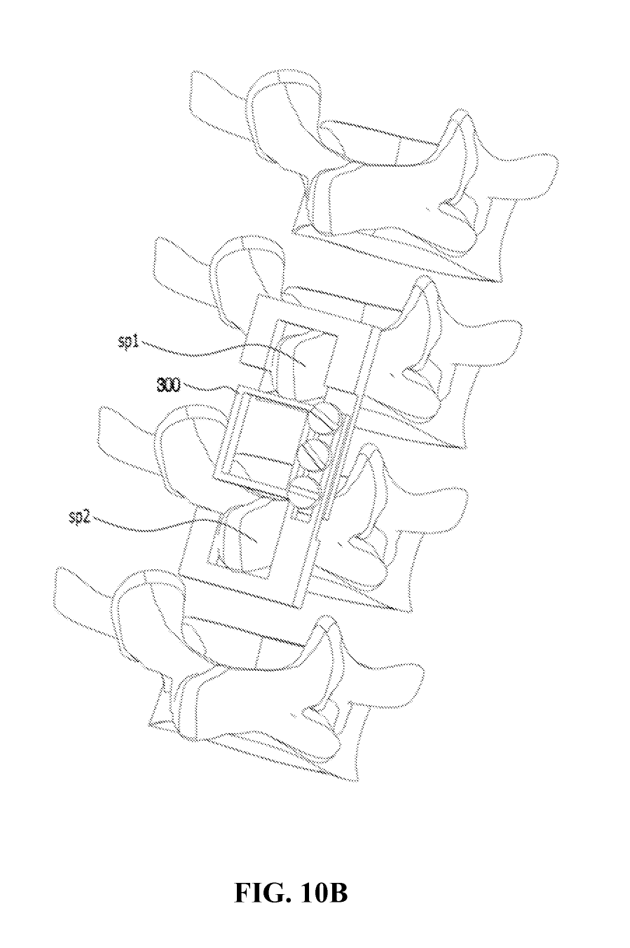

Referring to FIG. 10a, the spacer 300 may have a penetrated shape so that an autogenous bone or artificial bone is filled therein to realize synostosis. When the autogenous bone or artificial bone is filled into the spacer 300, the implant may be effectively fused between the spinous processes.

The upper hook 110 and the lower hook 210 include a first protrusion (not shown) and a second protrusion (not shown) which protrude from surfaces opposite to those on which the insertion part 130 and the guide frame 230 are disposed, respectively. Through holes 410 and 430 defined in both ends of the plate 400 may be press-fit coupled to the first and second protrusions and hooked between the adjacent spinous processes sp1 and sp2 to more safely fix the fixed upper and lower hooking members 100 and 200. The first and second protrusions have structures that protrude from one side surface of each of the upper and upper hooks 110 and 210, unlike first and second coupling pins that will be described below.

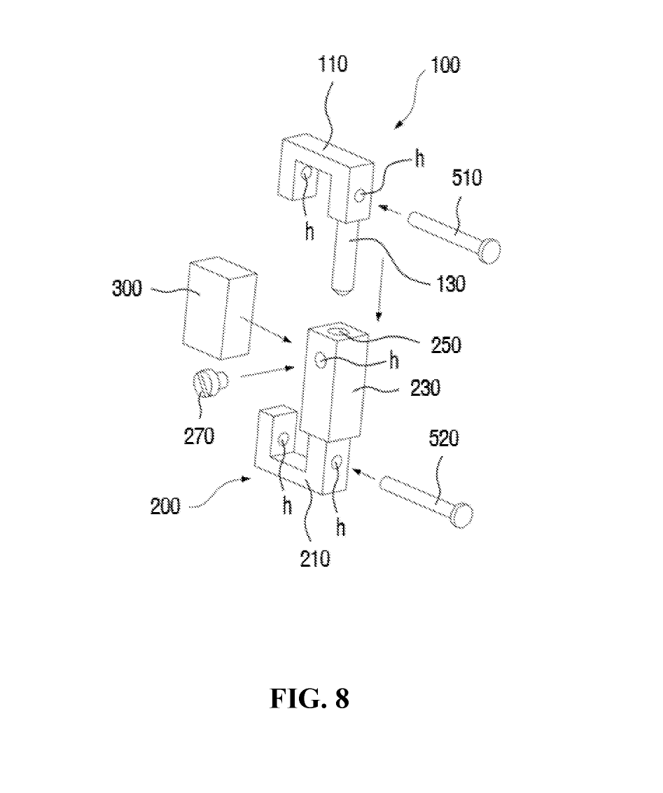

Referring to FIGS. 7A and 8, the interspinous fusion implant further includes first and second coupling pins 510 and 520 each of which horizontally passes through holes h defined in both side surfaces of each of the upper and lower hooks 110 and 210 to protrude toward a surface (that is the other surface on which each of the insertion part 130 and the guide frame 230 is not disposed) opposite to that on which each of the insertion part 130 and the guide frame 230 is disposed. The first coupling pin 510 passes through the upper spinous process sp1, to which the upper hook 110 is hooked, to protrude to an opposite surface, and the second coupling pin 520 passes through the lower spinous process sp2, to which the lower hook 210 is hooked, to protrude to an opposite surface. Since the first and second coupling pins 510 and 520 are coupled to screw threads disposed on inner circumferential surfaces of the holes h that are defined in the surfaces opposite to each other, respectively, the first and second coupling pins 510 and 520 may be stably coupled without being separated.

Referring to FIGS. 7B and 8, the interspinous fusion implant further includes a plate 400 that is press-fit coupled to the first and second coupling pins 510 and 520, which protrude to the opposite surfaces, to fix the upper and lower hooking members 100 and 200. The through holes 410 and 430 defined in the plate 400 are coupled to the first and second coupling pins 510 and 520, respectively. Since the plate 400 is coupled to the first and second coupling pins 510 and 520, the upper and lower hooking members 100 and 200 may be more stably fixed.

Each of the upper and lower hooking members 100 and 200 may have various sizes and lengths because the insertion part 130 is slidably coupled to the guide frame 230 to constitute the whole implant for each patient during an actual operation. Thus, the plate 400 may have various sizes.

According to the above-described second embodiment, when the upper and lower hooking members 100 and 200 are coupled to each other, each of the upper and lower hooking members 100 and 200 may only linearly move to be firmly fixed through the fastening member. Thus, the operation may be more simply performed to minimize a patient's side effect when compared to the first embodiment.

### Third Embodiment

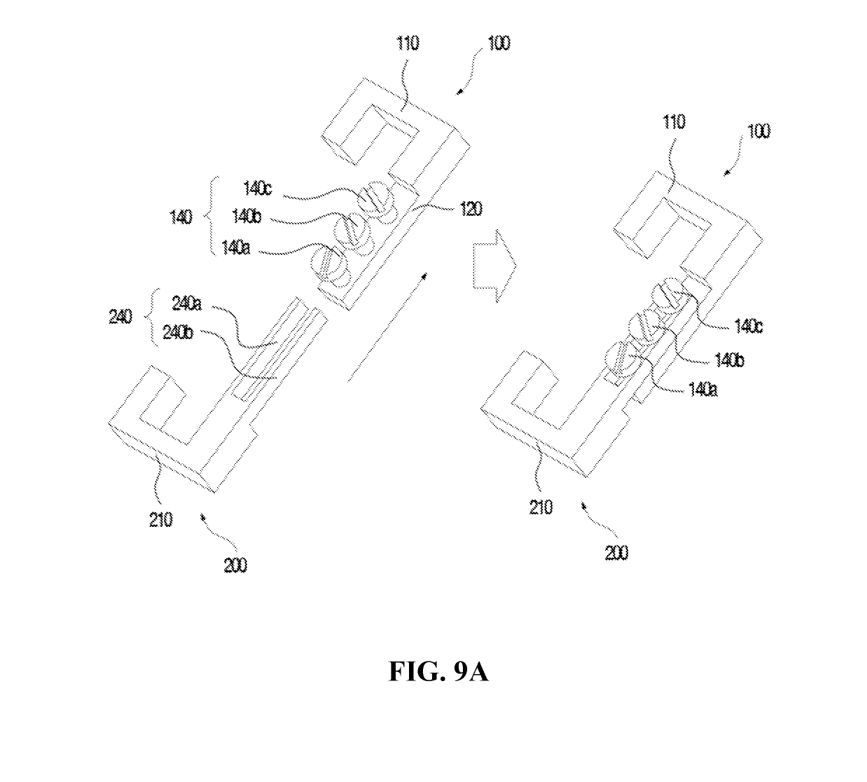

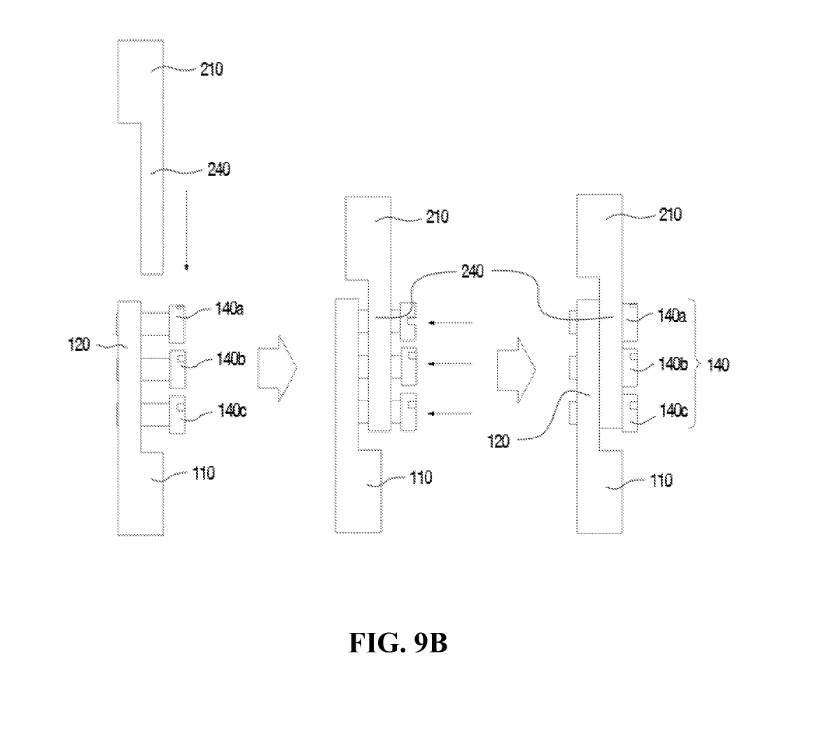

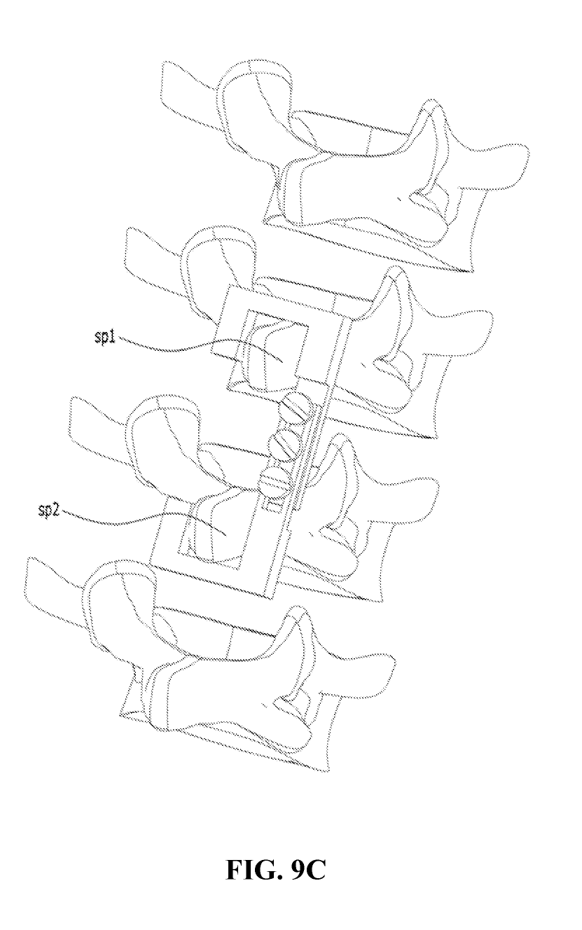

Referring to FIGS. 9A to 10B, an interspinous fusion implant according to a second embodiment includes an upper hooking member 100 hooked with an upper spinous process sp1 of a spine having a lesion and a lower hooking member 200 hooked with an adjacent lower spinous process sp2, wherein gapping of the upper and lower spinous processes sp1 and sp2 in opposite directions is prevented. The upper hooking member 100 includes an upper hook 110 for pulling the upper spinous process sp1 downward, a first coupling part 120 that is recessed to be stepped downward by a predetermined length at one side of the upper hook 100, and a plurality of fastening members 140 inserted in a line along a longitudinal direction of the first coupling part 120, and the lower hooking member 200 includes a lower hook 210 for pulling the lower spinous process sp2 upward and a second coupling part 240 that is recessed to be stepped upward by a predetermined length at one side of the lower hook 210, wherein a slot-shaped accommodation groove is defined in a center of the second coupling part 240 along a longitudinal direction to divide the second coupling part 240 into two parts (left and right parts 240a and 240b). The second coupling part 240 is guided by the plurality of fastening members 140 successively coupled to the accommodation groove having the slot shape and then engaged with the first coupling part 120. In the current embodiment, the shapes of the upper and lower hooks 110 and 210 are not specifically limited.

Each of the plurality of fastening members 140 includes a body press-fit coupled to the first coupling part 120 and a head disposed on an upper portion of the body. After the bodies of the fastening member 140 are successively accommodated into the accommodation groove of the second coupling part 240 and coupled, when the plurality of fastening members 140 are pushed to be closely attached, the second coupling part 240 may be fixed by the heads without being separated. The second coupling part 240 is fitted between the head and the first coupling part 120. Although a total of three fastening members 140 are illustrated in the drawings, the present disclosure is not limited thereto. For example, at least two fastening members 140 may be provided. The head and body of the fastening member 140 may be similar to a head and body of a bolt, respectively.

Referring to FIGS. 10A and 10B, the interspinous fusion implant further includes a spacer 300 that is parallely attached to a side surface of the second coupling part 240 and disposed between the upper and lower spinous processes sp1 and sp2 to maintain a minimum distance between the spinous processes sp1 and sp2. The spacer 300 has a penetrated shape so that an autogenous bone or artificial bone is filled therein to realize synostosis. Since the spacer 300 was described in detail in the second embodiment, a detailed description thereof will not be provided again.

The upper hook 110 and the lower hook 210 include a first protrusion (not shown) and a second protrusion (not shown) which protrude from surfaces opposite to those on which the first and second coupling parts 120 and 240 are disposed, respectively. The interspinous fusion implant further includes the plate that is press-fit coupled to the first and second protrusions to fix the upper and lower hooking members 100 and 200. Since the plate was described in detail in the second embodiment, a detailed description thereof will not be provided again.

The interspinous fusion implant further includes first and second coupling pins each of which horizontally passes through holes h defined in both side surfaces of each of the upper and lower hooks 110 and 210 to protrude toward a surface (that is the other surface on which each of the first and second coupling parts 120 and 240 is not disposed) opposite to that on which each of the first and second coupling parts 120 and 240 is disposed. A first coupling pin passes through the upper spinous process sp1, to which the upper hook 110 is hooked, to protrude to an opposite surface, and a second coupling pin passes through the lower spinous process sp2, to which the lower hook 210 is hooked, to protrude to an opposite surface. Since the first and second coupling pins are coupled to screw threads disposed on inner circumferential surfaces of the holes h that are defined in the surfaces opposite to each other, respectively, the first and second coupling pins may be stably coupled without being separated.

The interspinous fusion implant further includes a plate that is press-fit coupled to the first and second coupling pins, which protrude to the opposite surfaces, to fix the upper and lower hooking members 100 and 200. Since the plate is coupled to the first and second coupling pins, the upper and lower hooking members 100 and 200 may be more stably fixed.

Although the first and second coupling pins and the plate are not shown in the third embodiment, the first and second coupling pins and the plate may have the same structure and be coupled through the same coupling method as those of the first and second coupling pins 510 and 520 and the plate 400 according to the second embodiment. Thus, since the structures and the coupling methods of the first and second coupling pins and the plate are described in the second embodiment, their detailed descriptions will be omitted.

Also, the interspinous fusion implant further includes a plate that is press-fit coupled to the first and second coupling pins to fix the upper and lower hooking members 100 and 200. Since the plate was described in detail in the second embodiment, a detailed description thereof will not be provided again.

According to the above-described third embodiment, when the upper and lower hooking members 100 and 200 are coupled to each other, each of the upper and lower hooking members 100 and 200 may only linearly move to be firmly fixed through the fastening member. Thus, the operation may be more simply performed to minimize a patient's side effect.

### Fourth Embodiment

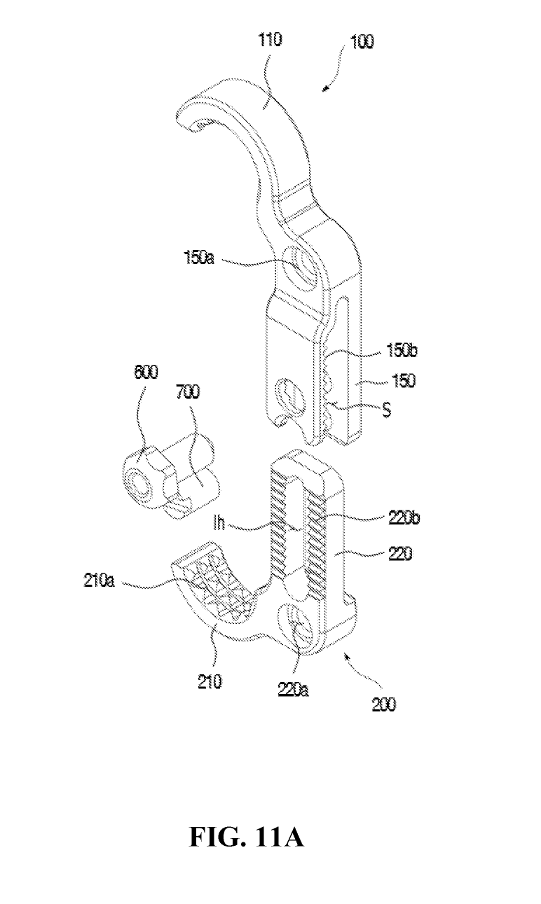

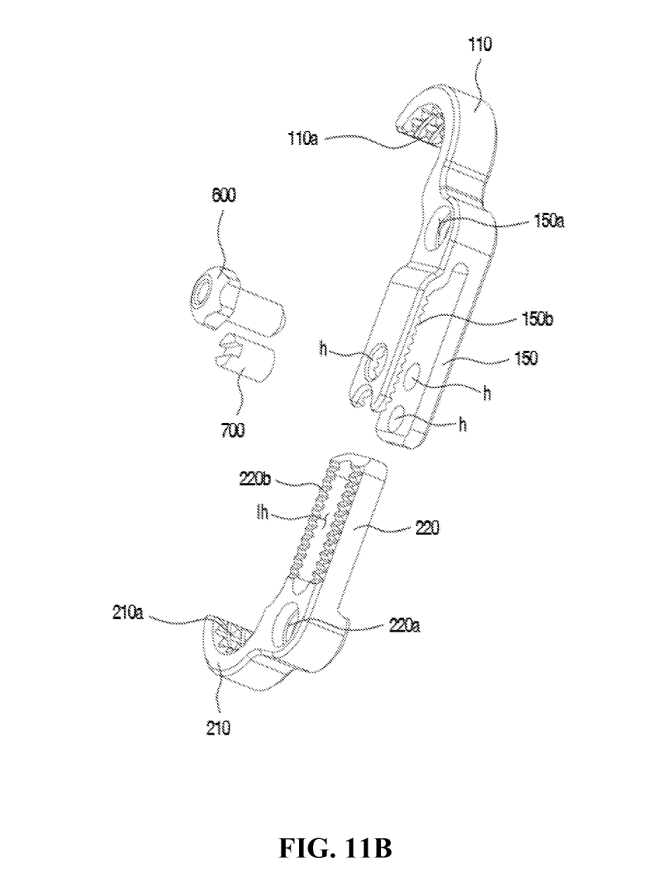

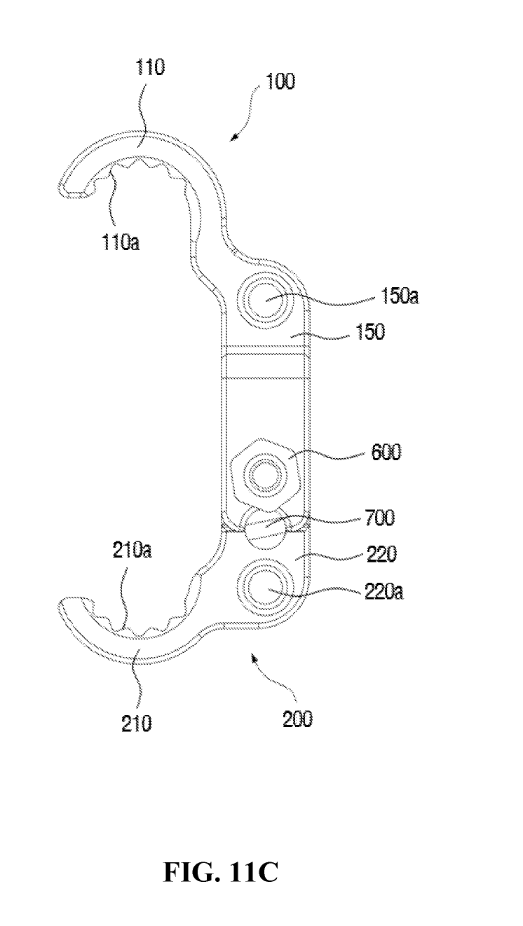

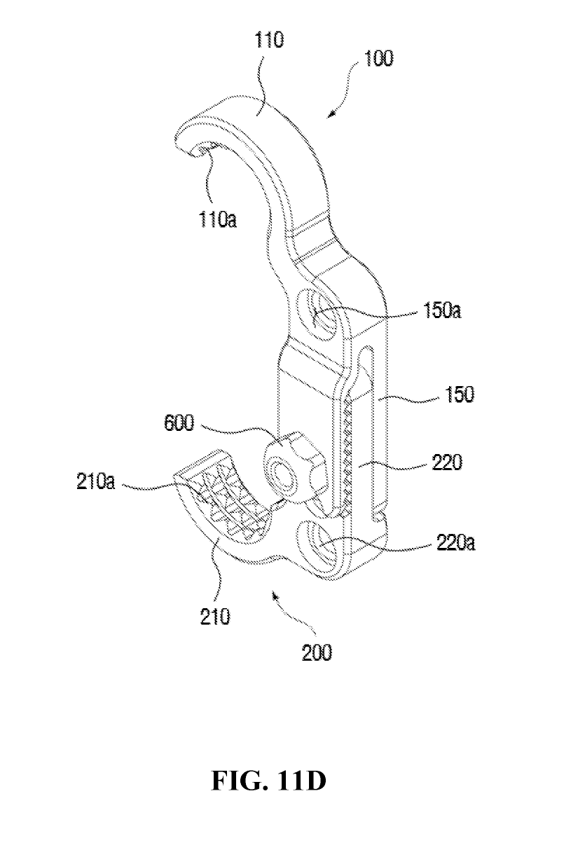

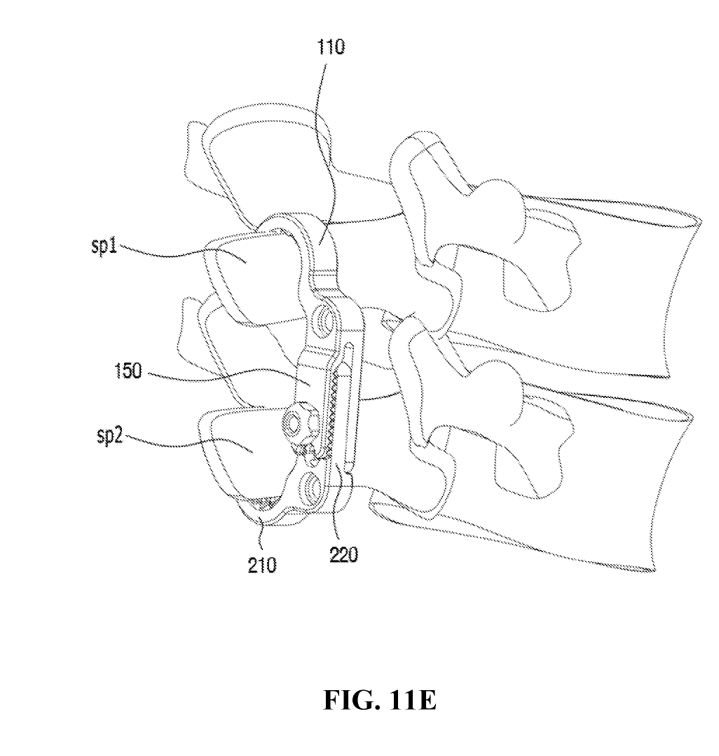

Referring to FIGS. 11A to 11E, an interspinous fusion implant according to a fourth embodiment includes an upper hooking member 100 hooked with an upper spinous process sp1 of a spine having a lesion and a lower hooking member 200 hooked with an adjacent lower spinous process sp2, wherein gapping of the upper and lower spinous processes sp1 and sp2 in opposite directions is prevented. The upper hooking member 100 includes an upper hook 110 for pulling the upper spinous process sp1 downward and an upper body 150 extending by a predetermined length from a lower portion of the upper hook 110 and having a slot S to which the lower hooking member 200 is coupled therein, and the lower hooking member 200 includes a lower hook 210 for pulling the lower spinous process sp2 upward and a lower body 220 extending by a predetermined length from an upper portion of the lower hook 210, having a long hole lh in a longitudinal direction of the body, and coupled to the slot S of the upper body 150. The interspinous fusion implant includes a fixing bolt 600 passing through the upper body 150 and the long hole lh of the lower body 220 coupled to the slot S of the upper body 150 and then coupled and fastened to fix the upper and lower hooking members 100 and 200 and a rotation preventing member 700 disposed on the same line as the fixing bolt 600 along a longitudinal direction of the upper body 150 to pass through the long hole lh of the lower body 220 together with the fixing bolt 600 and to be coupled to prevent the coupled lower body 220 from rotating with respect to the fixing bolt 600 as an axis and allow the lower body 220 to linearly move when the coupled lower body 220 moves within the slot S. A hole h through which the fixing bolt 600 passes is defined in the upper body 150, and also another hole h or groove to which the rotation preventing member 700 is coupled is defined in the upper body 150. Here, the rotation preventing member 700 may be coupled to be inserted into the groove or to pass through the hole h.

Although the rotation movement of the lower hooking member 200 occurs when the lower hooking member 200 moves because the fastening of the lower hooking member 200 and the guiding of the movement of the lower hooking member 200 are realized by using one fixing bolt 30 in the first embodiment, in the fourth embodiment an auxiliary unit such as the rotation preventing member 700 in addition to the fixing bolt 600 may be provided to fundamentally restrict the rotation movement of the lower hooking member 200, through which both the fixing bolt 600 and the rotation preventing member 700 pass to be coupled, and prevent an unnecessary motion from occurring, thereby more quickly performing a surgical operation and preventing neural damage around a surgical site from occurring. The rotation preventing member 700 is coupled to the groove or hole h that is defined in a lower end of the upper body 150. The rotation preventing member 700 does not have a bolt shape and has a length less than that of the fixing bolt 600. Also, the fixing bolt 600 is disposed at a position adjacent to the rotation preventing member 700.

When the rotation preventing member 700 is disposed on the lower end of the upper body 150, and the fixing bolt 600 is disposed at a position adjacent to the rotation preventing member 700, the lower hooking member 200 may move out of the slot S of the upper body 150 to maximally and sufficiently elongate to easily remove the implant from the adjacent spinous process. In addition, since the implant is reduced in distance after the implant elongates first when the implant is hooked with the upper and lower spinous processes sp1 and sp2, the above-described structure has to be provided.

If the fixing bolt 600 is disposed to be spaced a predetermined distance from the rotating preventing member 700, or the rotation preventing member 700 is disposed above the fixing bolt 600, the distance by which the lower hooking member 200 moves into the slot S may be restricted. As a result, many inconveniences may occur during the surgical operation.

The fixing bolt 600 and the rotation preventing member 700 may have the same diameter. The long hole lh defined in the lower body 220 may have a horizontal width that is enough to allow the fixing bolt 600 and the rotation preventing member 700 to pass therethrough.

In addition, upper and lower hooking holes 150a and 220a are defined in the upper and lower bodies 150 and 220, respectively.

The upper and lower hooking holes 150a and 220a may be used for respectively hooking the upper and lower hooking members 100 and 200 with the upper and lower spinous processes sp1 and sp2 by coupling a separate mechanism thereto and also for reducing the distance between the hooked upper and lower hooking members 100 and 200.

The upper and lower hooking holes 150a and 220a may not be necessarily defined in the upper and lower hooking members 100 and 200, respectively. For example, for convenience during surgery, the upper and lower hooking holes 150a and 220a may be selectively defined by a manufacturer.

An upper latch 150b is disposed inside the upper body 150 of the upper hooking member 100, and a lower latch 220b is disposed on one surface of the lower body 220 of the lower hooking member 200. When the fixing bolt 600 is fastened after the lower hooking member 200 is coupled to the upper hooking member 100, the upper and lower bodies 150 and 220 are more closely attached to each other. Here, since the upper and lower latches 150a and 220b are engaged with each other, the upper and lower bodies 150 and 220 may be stably coupled to each other.

A plurality of protrusions 110a and 210a are disposed on inner surfaces of the upper and lower hooks 110 and 210 to allow the upper and lower hooks 110 and 210 to be stably hooked with the upper and lower spinous processes sp1 and sp2 without slipping.

According to the above-described fourth embodiment, when the upper and lower hooking members 100 and 200 are coupled to each other, each of the upper and lower hooking members 100 and 200 may only linearly move to be firmly fixed through the fixing bolt without performing an unnecessary rotation movement. Thus, the surgical operation may be more simply performed to minimize a patient's side effect when compared to the first embodiment.

As described above, since the interspinous fusion implant is simply hooked with the spinous process, unlike the existing method in which an implant is fixed to the pedicle by using the pedicle screw, the procedural time may be reduced, and also, limitations arising due to the pedicle screw may be fundamentally prevented.

In addition, since the procedure is performed in the state in which the skin is minimally incised, the patient may be quickly recovered.

Furthermore, when the upper hooking member and the lower hooking member are pulled toward each other so that the upper and lower hooking members are closely attached to each other in the state in which the upper hooking member is hooked with one spinous process of the portion having the lesion, and the lower hooking member coupled to the upper hooking member is hooked with the other adjacent spinous process, the upper and lower hooking members may be closely attached to each other through the linear movement without the unnecessary operation such as the rotation of the lower hooking member when the upper and lower hooking members are initially coupled to each other. Thus, the operation time may be more reduced, and the occurrence of the limitation such as the neural damage around the surgical site may be prevented.

According to the embodiments, since the upper hooking member and the lower hooking member are pulled toward each other so that the upper and lower hooking members are closely attached to each other in the state in which the upper hooking member is hooked with one spinous process of the portion having the lesion, and the lower hooking member coupled to the upper hooking member is hooked with another adjacent spinous process to fix the hooked state by using the fixing bolt, the upper and lower hooking members that are respectively hooked with the spinous processes may be stably fixed, and the fixed state between the upper and lower hooking members may be stably maintained by the upper and lower latches that are respectively disposed on the upper and lower hooking members.

According to the embodiments, when the upper hooking member and the lower hooking member are pulled toward each other so that the upper and lower hooking members are closely attached to each other in the state in which the upper hooking member is hooked with one spinous process of the portion having the lesion, and the lower hooking member coupled to the upper hooking member is hooked with another adjacent spinous process, the upper and lower hooking members may be closely attached to each other through the linear movement without the unnecessary operation such as the rotation of the lower hooking member when the upper and lower hooking members are initially coupled to each other. Thus, the operation time may be reduced, and limitations such as neural damage around the surgical site may be prevented.

Also, after the upper and lower hooking members linearly move to be coupled to each other, the coupled state of the upper and lower hooking members may be fixed by using the fastening member or the fixing bolt. Thus, the upper and lower hooking members which are respectively hooked with the adjacent spinous processes may be stably fixed.

Also, the required minimum distance between the upper and lower spinous processes may be maintained by the spacer to prevent adjacent spinous processes from being unnecessarily closely attached to each other during a surgical operation, thereby improving stability in the surgical operation.

Furthermore, the autogenous bone or the artificial bone may be filled into the spacer having the perforated shape to realize synostosis, thereby improving operation effects.

Also, after the upper and lower hooking member are closely attached and coupled to each other, the upper and lower hooking members may be additionally fixed by using the plate to further improve operation stability and operation effects.

As described above, when the interspinous fusion implant is installed, the interspinous fusion implant may be hooked with the spinous process of the spine having the lesion through the minimum incision to prevent limitations due to the pedicle screw fixation, i.e., damage to the nerves due to the fixation using pedicle perforation from occurring.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An interspinous fusion implant comprising:
an upper hooking member installed to be hooked with one spinous process; and
a lower hooking member installed to be hooked with another adjacent spinous process,
wherein the upper hooking member and the lower hooking member are coupled to each other and then fixed to each other through a fixing bolt after a distance between vertebral bodies having a lesion is reduced by using a separate mechanism.

2. The interspinous fusion implant of claim 1, wherein the upper hooking member comprises:
an upper body having a “ [symbol] ” shape;
an upper latch disposed on one surface of the inside of the upper body to fix a position of the lower hooking member;
a fixing hole to which the fixing bolt is coupled to fix the upper hooking member and the lower hooking member;
a rotation preventing protrusion disposed inside the upper body to prevent the lower hooking member coupled to the upper hooking member from rotating; and
an upper hook integrated with one side of the upper body so as to be hooked with one spinous process.

3. The interspinous fusion implant of claim 1, wherein the lower hooking member comprises:
a lower body movably and rotatably installed inside the upper body;
a lower latch disposed on one surface of the inside of the lower body so that the lower latch is engaged with the upper latch and fixed in position;
a long hole defined in the lower body so that the long hole is coupled to the fixing bolt to allow the lower body to move and rotate inside the upper body; and
a lower hook integrated with one side of the lower body so that the lower hooking member is hooked with the other spinous process that is adjacent to the spinous process with which the upper hooking member is hooked.

4. The interspinous fusion implant of claim 1, wherein the fixing bolt inserted into the upper hooking member and coupled to the long hole of the lower hooking member and the fixing hole of the upper hooking member is inserted into and coupled to the lower hooking member, and
the fixing bolt is supported on one side of the long hole of the lower hooking member that is inserted into and coupled to the inside of the upper hooking member to rotate or move in a longitudinal direction of the upper hooking member.

5. An interspinous fusion implant comprising:
an upper hooking member hooked with an upper spinous process; and
a lower hooking member hooked with an adjacent lower spinous process,
wherein gapping of the upper and lower spinous processes in opposite directions is prevented,
the upper hooking member comprises an upper hook for pulling the upper spinous process downward and an insertion part disposed with a predetermined length on a lower portion of the upper hook, and
the lower hooking member comprises a lower hook for pulling the lower spinous process upward, a guide frame disposed on an upper portion of the lower hook and in which the insertion part is slidably coupled to an inner accommodation part that is longitudinally defined therein, and a fastening member passing from one side surface of the guide frame and coupled to the guide frame, the fastening member being closely attached to the insertion part coupled to the inner accommodation part to firmly fasten the insertion part.

6. The interspinous fusion implant of claim 5, wherein an inner circumferential surface of the guide frame has a circular or oval shape, and
an outer circumferential surface of the insertion part has a circular shape.

7. The interspinous fusion implant of claim 5, wherein an inner circumferential surface of the guide frame and an outer circumferential surface of the insertion part have polygonal shapes corresponding to each other.

8. The interspinous fusion implant of claim 5, further comprising a spacer parallely attached to a side surface of the guide frame and disposed between the upper and lower spinous processes to maintain a minimum distance between the spinous processes.

9. The interspinous fusion implant of claim 8, wherein the spacer has a penetrated shape so that an autogenous bone or artificial bone is filled therein for synostosis.

10. The interspinous fusion implant of claim 5, wherein the upper and lower hooks comprise first and second protrusions which protrude from surfaces opposite to those on which the insertion part and the guide frame are disposed, respectively, and
the interspinous fusion implant further comprises a plate press-fit coupled to the first and second protrusions to fix the upper and lower hooking members.

11. The interspinous fusion implant of claim 5, further comprising first and second coupling pins each of which horizontally passes through and is coupled to both side surfaces of each of the upper and lower hooks to protrude toward a surface opposite to that on which each of the insertion part and the guide frame is disposed,
wherein the first coupling pin passes through the upper spinous process to protrude to the opposite surface, and
the second coupling pin passes through the lower spinous process to protrude to the opposite surface.

12. The interspinous fusion implant of claim 11, further comprising a plate press-fit coupled to the first and second coupling pins which respectively protrude to the opposite surfaces to fix the upper and lower hooking members.

13. An interspinous fusion implant comprising:
an upper hooking member hooked with an upper spinous process; and
a lower hooking member hooked with an adjacent lower spinous process,
wherein gapping of the upper and lower spinous processes in opposite directions is prevented,
the upper hooking member comprises an upper hook for pulling the upper spinous process downward, a first coupling part that is recessed to be stepped downward by a predetermined length at one side of the upper hook, and a plurality of fastening members inserted in a line along a longitudinal direction of the first coupling part,
the lower hooking member comprises a lower hook for pulling the lower spinous process upward and a second coupling part that is recessed to be stepped upward by a predetermined length at one side of the lower hook, wherein a slot-shaped accommodation groove is defined in a center of the second coupling part along a longitudinal direction to divide the second coupling part into a left and a right part, and
the second coupling part is guided by the plurality of fastening members coupled in sequence to the slot-shaped accommodation groove, and is engaged with the first coupling part.

14. The interspinous fusion implant of claim 13, wherein each of the plurality of fastening members comprises a body press-fit coupled to the first coupling part and a head disposed on an upper portion of the body, and
when the plurality of fastening members are pushed to be closely attached after the bodies of the fastening members are successively accommodated into and coupled to the accommodation groove of the second coupling part, the second coupling part is fixed by the heads without being separated.

15. The interspinous fusion implant of claim 13, further comprising a spacer parallely attached to a side surface of the second coupling part and disposed between the upper and lower spinous processes to maintain a minimum distance between the spinous processes.

16. The interspinous fusion implant of claim 15, wherein the spacer has a penetrated shape so that an autogenous bone or artificial bone is filled therein for synostosis.

17. The interspinous fusion implant of claim 13, wherein the upper and lower hooks comprise first and second protrusions which protrude from surfaces opposite to those on which the first and second coupling parts are disposed, respectively, and
the interspinous fusion implant further comprises a plate press-fit coupled to the first and second protrusions to fix the upper and lower hooking members.

18. The interspinous fusion implant of claim 13, further comprising first and second coupling pins each of which horizontally passes through and is coupled to both side surfaces of each of the upper and lower hooks to protrude to a surface opposite to that on which each of the first and second coupling parts is disposed,
wherein the first coupling pin passes through the upper spinous process to protrude to the opposite surface, and
the second coupling pin passes through the lower spinous process to protrude to the opposite surface.

19. The interspinous fusion implant of claim 18, further comprising a plate press-fit coupled to the first and second coupling pins which respectively protrude to the opposite surfaces to fix the upper and lower hooking members.

20. An interspinous fusion implant comprising:
an upper hooking member hooked with an upper spinous process; and
a lower hooking member hooked with an adjacent lower spinous process,
wherein gapping of the upper and lower spinous processes in opposite directions is prevented,
the upper hooking member comprises an upper hook for pulling the upper spinous process downward and an upper body extending by a predetermined length from a lower portion of the upper hook and having a slot into which the lower hooking member is coupled,
the lower hooking member comprises a lower hook for pulling the lower spinous process upward and a lower body extending by a predetermined length from an upper portion of the lower hook, having a long hole in a longitudinal direction of the body, and coupled to the slot of the upper body,
wherein the interspinous fusion implant further comprises:
a fixing bolt passing through the upper body and the long hole of the lower body coupled to the slot of the upper body and coupled and fastened to fix the upper and lower hooking members; and
a rotation preventing member disposed on the same line as the fixing bolt along a longitudinal direction of the upper body to pass through the long hole of the lower body together with the fixing bolt and to be coupled to prevent the coupled lower body from rotating with respect to the fixing bolt as an axis and allow the lower body to linearly move when the coupled lower body moves within the slot of the upper body.

21. The interspinous fusion implant of claim 20, wherein the rotation preventing member is coupled to a lower end of the upper body.

22. The interspinous fusion implant of claim 20, wherein the long hole defined in the lower body has a horizontal width that is enough to allow the fixing bolt and the rotation preventing member to pass therethrough.
